(19) United States
(12) Patent Application Publication
(10) Pub. No.: US 2004/0058975 A1
(43) Pub. Date: Mar. 25, 2004

(54) Method for fertility control

(75) Inventors: Bernhard Lindenthal, Berlin (DE); Bernd Buchmann, Hohen Neuendorf (DE); Werner Skuballa, Berlin (DE); Christa Hegele-Hartung, Mulheim a. d. Ruhr (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/606,289

(22) Filed: Jun. 26, 2003

Related U.S. Application Data
(60) Provisional application No. 60/414,363, filed on Sep. 30, 2002.

(30) Foreign Application Priority Data
Jun. 26, 2002 (GB) 0214802.1

(51) Int. Cl. A61K 31/415; A61K 31/365

(52) U.S. Cl. 514/406; 514/471

(57) ABSTRACT

The present invention relates to a method for impairing cumulus expansion and oocyte maturation, the method comprising antagonizing EP2 receptor and/or inhibiting cyclooxygenase COX-2. The invention also relates to the use of a pharmaceutical composition comprising an EP₂ receptor antagonist (optionally with one or more COX inhibitors) for female contraception.

This application claims the benefit of the filing date of U.S. Provisional Application Ser. No. 60/414, 363 filed Sep. 30, 2002.

The present invention relates to a new method for fertility control.

### BACKGROUND TO THE INVENTION

Current methods of fertility control include physical prevention of gamete interaction by barrier methods, spermicides, surgical methods, and hormonal disruption of the menstrual cycle using steroids (Hsueh, 1995). The latter strategy, commonly known as the steroid hormone contraceptive pill, is one of the most widely used methods of contraception by women. Although of undisputed efficacy, the long-term exposure to oestrogen/progestogen means that this method involves several undesirable side effects. Such side effects may include a possible increase in the incidence of breast cancer and thrombosis, risks which have been the focus of concern and public debate. Whilst recent epidemiological data point towards a trend for the improved tolerability of low-dose hormonal preparations in respect of cardiovascular side effects [Thorogood M, Oral Contraceptives and Cardiovascular Disease: an Epidemiologic Overview; Pharmacoepidemiology and Drug Safety, Vol. 2: 3-16 (1993); Gerstman B. B., Piper J. M., Tomita D. K., Ferguson W. J., Stadel B. V., Lundin F. E.; Oral Contraceptive Estrogen Dose and the Risk of Deep Venous Thromboembolic Disease, Am. J. E., Vol.133, No. 1, 32-36 (1991); Lidegaard O., Oral contraception and the risk of a cerebral thromboembolic attack: results of a case-control study; BMJ Vol. 306, 956-63 (1993); Vessey M., Mant D., Smith A., Yeates D., Oral contraceptives and venous thromboembolism: findings in a large prospective study; BMJ, Vol. 292 (1986); Mishell D. R., Oral Contraception: Past, Present and Future Perspectives; Int. J. Fertil., 36 Suppl., 7-18 (1991)], there exists a need for a safe, effective and reliable method of contraception that does not have the undesirable side effects associated with steroid hormone contraceptives.

### SUMMARY OF THE INVENTION

The invention relates to a method for imparing cumulus expansion and oocyte maturation, the method comprising antagonizing a prostaglandin type 2 (EP₂) receptor and/or inhibiting cyclooxygenase COX-2.

The present invention also relates to compositions comprising an EP₂ receptor antagonist and optionally a COX inhibitor capable of modulating EP₂ receptor activity for use in contraception.

An important finding of the present invention is the provision of a non-steroid hormonal composition for use in contraception based on the demonstration that inhibition of prostaglandin action on late folliculogenesis prevents ovulation and/or the production of fertilisable oocytes.

Surprisingly, we have found that the processes of oocyte maturation and ovulation can be uncoupled using inhibitors of the prostaglandin type 2 (EP₂) receptor involved in folliculogenesis.

In particular, we have found that EP₂ antagonists may impair cumulus expansion and oocyte maturation preventing ovulation completely or delaying release of an oocyte which may not have the competence to be fertilised. Thus, such compounds may be considered contraceptive agents.

An advantage of these findings is the prevention of oocyte maturation and subsequent fertilisation without disruption of ovulation and of the normal menstrual cycle. An endogenous hormonal environment is therefore maintained.

The present invention thus provides a non-steroid hormone application that is an alternative to current conventional steroid oral contraceptive methods.

### DETAILED DISCLOSURE OF THE INVENTION

Aspects of the present invention are presented in the accompanying claims and in the following description.

By way of example, key aspects of the present invention relate to:

A method for imparing cumulus expansion and oocyte maturation, the method comprising antagonizing EP₂ receptor and/or inhibiting cyclooxygenase COX-2.

A method, wherein the EP₂ receptor is antagonised by the EP₂ receptor antagonist AH6809.

A method, wherein said EP₂ receptor antagonist is used in a composition and wherein said composition further comprises one or more COX inhibitors.

Use of an EP₂ receptor antagonist as contraceptive.

Use of an EP₂ receptor antagonist (optionally in combination with one or more COX inhibitors) in the preparation of a contraceptive in an amount effective to prevent ovulation and/or the production of fertilisable oocytes.

Use of an EP₂ receptor antagonist (optionally in combination one or more COX inhibitors) in the preparation of a contraceptive in an amount effective to delay folliculogenesis and/or follicle rupture.

Use of an EP₂ receptor antagonist (optionally in combination with one or more COX inhibitors) in the preparation of a contraceptive in an amount effective to inhibit cumulus expansion in pre-ovulatory follicles.

A pharmaceutical composition comprising a therapeutically effective amount of an EP₂ receptor antagonist (optionally in combination with one or more COX inhibitors) admixed with a pharmaceutically acceptable carrier, diluent or excipient (including combinations thereof).

A pharmaceutical composition for simultaneous, separate or sequential administration to a subject in need thereof comprising an EP₂ receptor antagonist either with one or more COX inhibitors and/or one or more other pharmaceutically active agents.

An in vitro assay for identifying putative EP₂ receptor antagonists comprising the use of cumulus enclosed oocyte (CEO) complexes.

An ex vivo assay for identifying putative EP₂ receptor antagonists comprising the use of ovarian perfusion assays.

An in vivo assay for identifying putative EP₂ receptor antagonists comprising administration of a putative EP₂ antagonist to a sexually mature or immature subject and measuring the resultant effects therein on ovulation, cumulus expansion, fertilisation, implantation and pregnancy rate.

An EP₂ receptor antagonist identified by the assay according to any one of the assays.

In the following commentary, reference to “an EP₂ receptor antagonist” refers to any one or more EP₂ receptor antagonists used either alone or in combination in the contraceptive compositions discussed herein.

Preferable aspects are presented in the accompanying claims and in the following description and Examples section.

Suitably the EP₂ receptor antagonist for the method for contraception and the use in the present invention may directly or indirectly modulate EP₂ receptor activity.

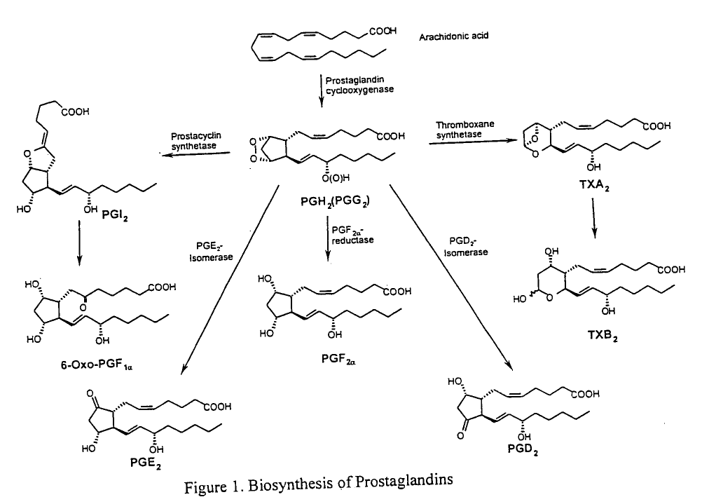

Prostaglandins (PG) are Prostanoids and belong to a family of oxidized metabolites of arachidonic acid (FIG. 1), they exhibit various physiological activities in order to maintain local homeostasis in organisms (The Pharmacological Basis of Therapeutics (Gilman, A. G., Goodman, L. S., Rall, T. W., and Murad, F., eds, 7th Ed., pp 660, Macmillan Publishing Co., New York (1985)). These physiological activities are controlled by a cell membrane receptor specific for each prostanoid (Annu. Rev. Pharm. Tox., 10, 213 (1989); Sugimoto et al, Prog.Lipid Res., vol. 39, pages 289-314, (2000); Narumiya et al, Physiol.Rev., vol. 79, pages 1193-1226, (1999) and ‘Prostanoids and their Receptors’ in Comprehensive Medicinal Chemistry, pp 643 (1990), Pergamon Press, Oxford).

The activity of prostaglandin PGE₂ is effected on a number of physiological and pathophysiological events in many tissues of the body. These effects are mediated through interaction with specific membrane-bound G protein-coupled prostanoid EP receptor.

EP 0,557,966 teaches a prostaglandin receptor which binds to prostaglandin subtype E₂ (PGE₂), (Coleman et al. 1994).

In addition to the biological effects mentioned it is also thought that prostaglandins, play an important role in reproduction, mediating effects such as increasing vascular permeability and activation and/or regulation of proteolytic enzymes (Marks & Furstenberger, 1999). For example, Higuchi et al (Prostaglandins, vol.49, pages 131-140 (1995)) have shown that ovarian PGE₂ levels are at a maximum prior to ovulation. More recently, Duffy and Stouffer (2001) have reported increased COX-2 expression and PGE₂ concentrations in follicular fluid prior to the expected time of ovulation in rhesus monkeys.

Recent analysis of knock-out mice for various prostaglandin receptors has revealed several events in the reproductive cycle which appear to be mediated via prostaglandin receptors. The development of EP₂ receptor knock-out mice has revealed the importance of the prostaglandin EP₂ receptor on late folliculogenesis, ovulation and fertilisation. For example, studies using knock-out mice have enabled the elucidation of the role of specific prostaglandin receptors in the reproductive cycle wherein undisturbed late folliculogenesis, including cumulus expansion of the cumulus oophorous (cumulus cells surrounding the oocyte) and oocyte maturation are a prerequisite for ovulation and subsequent fertilisation (Sugimoto et al, Prog.Lipid Res., vol.39, pages 289-314 (2000) and Challis, Nature Medicine, vol.3, pages 1326-1327 (1997))).

Further studies have shown a marked reduction in the fertility of EP₂ receptor knockout mice. Such sub-fertility has been attributed to anovulation or a-marked reduction in ovulated oocytes (Matsumoto et al, 2001), or markedly reduced in vivo fertilisation rates (Hizaki et al, 1999 and Tilly et al, 1999). Tilley et al. (1999) have also shown that mice deficient in the prostaglandin EP₂ receptor (Ep2-/- females) are infertile secondary to failure of the released ovum to become fertilized in vivo. Ep2-/- ova could be fertilized in vitro, suggesting that in addition to previously defined roles, prostaglandins may contribute to the microenvironment in which fertilisation takes place.

Hizaki et al 1999 have shown that mainly cumulus expansion in late folliculogenesis is impaired in EP₂ receptor knock-out mice.

An investigation of the EP₂ receptor knock-out mouse by Kennedy et al (1999) also suggests that EP₂ receptor activation plays an important role in ovulation and subsequent fertilisation processes. It has been reported therein that the “failure of EP₂ receptor activation may contribute to the reversible infertility associated with non-steroidal anti-inflammatory drug use (e.g. COX inhibitors).” The same authors have suggested that the “EP₂ receptor may thus prove to be a productive target for pharmacological intervention in the treatment of hypertension and infertility.” However, the exact mechanism is still under debate.

As used herein, the term “EP₂ antagonist” means any agent that directly or indirectly modulates EP₂ receptor activity.

Typically the effects of EP₂ receptor antagonists on the EP₂ receptor are manifested as a deactivation of EP₂ receptor activity.

Preferably the EP₂ receptor antagonist is selective. Thus according to one aspect of the present invention there is provided the use of an EP₂ receptor antagonist which when in use is selective, such as highly selective, for EP₂ receptors associated with reproduction, in particular, those associated with folliculogenesis, ovulation and fertilisation.

Preferably, the EP₂ receptor antagonists for use in the contraceptive compositions according to the present invention have an IC₅₀ of less than 500 nanomolar (nM), more preferably of less than 400 nM, more preferably of less than 300 nM, more preferably of less than 200 nM, more preferably of less than 100 nM.

The term “selective” as used herein means the EP₂ receptor antagonists according to the present invention have greater than about 5-fold, more preferably greater than about 10-fold, more preferably greater than about 25-fold, more preferably greater than about 50-fold selectivity for the EP₂ receptor over the other prostanoid receptors such as EP₁, EP₃, EP₄, DP, FP, IP or TP receptors. Preferably the EP₂ receptor antagonist has no, or substantially no, activity towards other prostanoid receptors such as EP₁, EP₃, EP₄, DP, FP, IP or TP receptors.

The term “highly selective” as used herein means the EP₂ receptor antagonists according to the present invention have greater than about 100-fold selectivity, preferably at least about 200-fold selectivity, preferably at least about 300-fold selectivity, preferably at least about 400-fold selectivity, preferably at least about 500-fold activity, preferably at least about 600-fold activity, preferably at least about 700-fold selectivity for EP₂ receptors.

An advantage of the use of EP₂ antagonists in the contraceptive compositions of the present invention is that oocyte function is blocked without interference of the reproductive hormonal milieu and the normal menstrual cycle. Thus, because the oestrous cycle and ovulation is not affected, the hypothalamic-pituitary-ovarian axis should not be altered during EP₂ receptor antagonist administration in vivo. This may provide an important advantage over certain side-effects associated with conventional steroid contraceptives.

The EP₂ receptor antagonist may be a chemical or biological molecule. The antagonist may be prepared by use of chemical synthetic technique or, if appropriate, by use of recombinant DNA techniques.

Suitable EP₂ receptor antagonists and synthetic routes thereto for use in the contraceptives of the present invention may be found in the art. By way of example, reference may be made to inter alia:

U.S. patent publication no. US2001005760;

U.S. provisional patent application no. 60/077,990

U.S. provisional patent application no. 60/103,564

U.S. provisional patent application no. 60/103,371

EP1175889

EP752421-Al

WO 00/024,393.

The disclosures of all are incorporated herein by reference.

Preferably, the EP₂ receptor antagonist for use in the contraceptive compositions of the present invention is AH6809 which is 6-isopropoxy-9-oxo-9H-xanthene-2-carboxylic acid, or a mimetic thereof.

General teachings on the EP₂ receptor antagonist AH6809 is provided in “Prostanoid Receptors: Structures, Properties, and Functions” Shuh Narumya et al., Physiological Review, 1999,1203-1204.

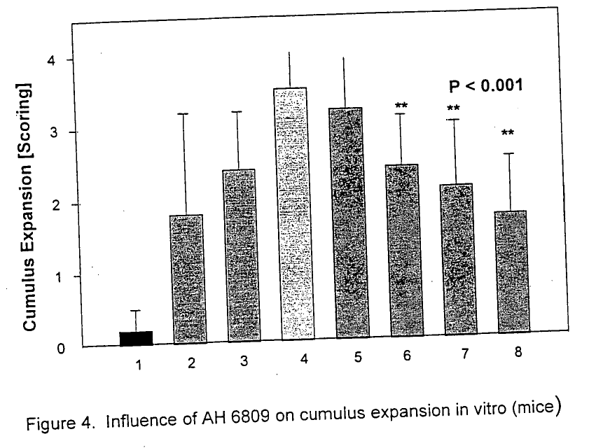

Deactivation of EP₂ receptor activity by an EP₂ antagonist directly or indirectly leads to impaired cumulus expansion and oocyte maturation in mice (FIG. 4). Thus, ovulation may be prevented or an oocyte may be released which does not have the competence to be fertilised and/or the release of which may be delayed.

In a preferred aspect the contraceptive compositions of the present invention comprise an EP₂ receptor antagonist in combination with one or more cyclooxygenase (COX) inhibitors.

The components of the composition may be for simultaneous, separate or sequential administration.

Cyclooxygenase enzymes exists in at least two different enzyme isoforms (Simmons et al., P.N.A.S. U.S.A. 86:1178-1182 (1989)), designated-COX-1 and COX-2 which metabolise arachidonic acid as an initial step for prostaglandin synthesisCOX-2 catalyses the synthesis of prostaglandins that cause inflammation and pain, but does not appear to catalyse prostaglandins. Both COX-1 and COX-2 are involved in producing precursors for several prostanoids including PGE₂. COX-1 is expressed constitutively at relatively stable levels in many tissues, whereas COX-2 expression can be induced by a variety of chemicals, including, but not limited to, lipopolysaccharides, phorbal esters, interleukin-1, tumour necrosis factor, human chorionic gonadotropin, and platelet activating factor. The various prostaglandins derived from COX activity may act in a paracrine or autocrine fashion via discrete receptors (Breyer et al, 2001).

The role of COX-2 in the reproductive cycle has become apparent with the use of COX-2 knock-out mice. For example, COX-2 has been shown to be inducible in response to gonadotropins in the rat ovary (Richards, 1994). COX-2 knock-out mice are infertile due to failings in ovulation, fertilisation, implantation and decidualisation (Lim et al., 1997), while mice lacking COX-1 expression have normal ovulation and fertilisation (Langenbach et al., 1995). Anovulation in COX-2 deficient mice is restored in the presence of prostaglandin E₂ and Interleukin-1β (Davis et al., 1999).

The reduction or prevention of ovulation by COX inhibitors has been demonstrated in several animal models (Murdoch et al, Prostglandins, vol.46, pages 85-115, 1993). For example, it has been reported that administration of non-selective COX inhibitors (NS-398; Aspirin, Naproxen, Indomethacin) may prevent ovulation in several mammalian species including rats (Mikuni et al., 1988), rabbits (Zanagnolo et al., 1996), and sheep (Murdoch, 1996). Furthermore, the administration of a non-selective COX inhibitor (Indomethacin) to monkeys (Wallach et al., 1975) and women (Killick and Elstein, 1987) has been shown to block ovulation. Recently Pall et al., (2001) demonstrated that selective COX-2 inhibition by Rofecoxib results in delayed follicular rupture, without affecting peripheral hormonal cycles in humans. In the rabbit, a selective COX-2 inhibitor (Meloxicam) has been demonstrated as an effective non-steroid hormonal contraceptive agent (Salhab et al. 2001).

However, inhibition of COX-2 activity may lead to the suppression of the synthesis of various prostaglandins in a non-specific manner thus increasing the probability of multiple mechanism-based side effects generally associated with COX-2 inhibitor treatments. Such side effects include gastrointestinal toxicity and irritation including upper gastrointestinal ulceration and bleeding and renal side effects such as reduction in renal function leading to fluid retention and exacerbation of hypertension and bleeding.

The cyclooxygenase inhibitor may be a chemical or biological molecule. The inhibitor may be prepared by use of chemical synthetic technique or, if appropriate, by use of recombinant DNA techniques.

Suitable cyclooxygenase inhibitors and synthetic routes thereto for use in the compositions of the present invention may be found in the art. By way of example, reference may be made to inter alia:

International Patent Publication No WO 95/00501

International Patent Publication No. WO 96/25405

International Patent Publication No. WO 97/38986

International Patent Publication No WO 98/034849

International Patent Publication No. WO 99/33794

International Patent Publication No. WO 01/41760

European Patent Application No. 0 799 823

European Patent Application No. 0 846 689

European Patent Application No. 0 863 134

European Patent Application No. 0 985 666

The disclosures of all are incorporated herein by reference.

Preferably, the COX inhibitor for use in the contraceptive compositions of the present invention is selected from the group consisting of Celecoxib which is 4-[5-(4-methylphenyl)-3-(trifluoromethyl)-1 -H-pyrazol-1-yl]benzenesulfonamide; Parecoxib which is N-[4-(5-methyl-3-phenyl-4-isoxazolyl)phenyl]sulfonylpropionamide; Rofecoxib which is 4-(4-mesylphenyl)-3-phenylfuran-2(5H)-one; Valdecoxib which is 4-[5-methyl-3-phenyl-4-isoxazoyl)benzenesulfonamide and NS-398 which is N-methyl-2-cyclohexanoxy-4-nitrobenzenesulfonamide, Ceracoxib and Etoricoxib.

More preferably the COX inhibitor for use in the contraceptive compositions of the present invention is Celecoxib or Rofecoxib.

Even more preferably the COX inhibitor for use in the contraceptive compositions of the present invention is Celecoxib.

Preferably the COX inhibitor is selective. Thus according to one aspect of the present invention there is provided the use of a COX inhibitor which when in use is selective, such as highly selective, for the cyclooxygenase-2 (COX-2) isoform.

Preferably, the COX inhibitors for use in the contraceptive compositions according to the present invention have an IC₅₀ of less than 500 nanomolar (nM), more preferably of less than 400 nM, more preferably of less than 300 nM, more preferably of less than 200 nM, more preferably of less than 100 nM.

The term “selective” as used herein means the COX inhibitors according to the present invention have greater than about 5-fold, more preferably greater than about 10-fold, more preferably greater than about 25-fold, more preferably greater than about 50-fold selectivity for the COX-2 isoform over other cyclooxygenase isoforms such as COX-1 isoform. Preferably the COX inhibitor has no, or substantially no, activity towards other cyclooxygenase isoforms such as COX-1.

The term “highly selective” as used herein means the COX inhibitors according to the present invention have greater than about 100-fold selectivity, preferably at least about 200-fold selectivity, preferably at least about 300-fold selectivity, preferably at least about 400-fold selectivity, preferably at least about 500-fold activity, preferably at least about 600-fold activity, preferably at least about 700-fold selectivity for cyclooxygenase-2 (COX-2) isoform.

The inhibition of EP₂ receptor-mediated processes by use of an EP₂ antagonist optionally in combination with one or more COX inhibitors in the contraceptive compositions of the present invention may provide an important advantage over multiple mechanism-based COX-2 inhibitor treatments which are associated with wide-ranging side effects.

The contraceptive compositions of the present invention may be used in combination with one or more other pharmaceutically active agents.

Thus, in one aspect, the present invention relates to a pharmaceutical composition for simultaneous, separate or sequential administration to a subject in need thereof comprising an EP₂ receptor antagonist (optionally in combination with one or more COX inhibitors) in combination with one or more other pharmaceutically active agents.

Suitable pharmaceutically active agents for use in the compositions of the present invention include:

Typically, the pharmaceutically active agent for use in the contraceptive compositions of the present invention may itself have contraceptive activity.

Pharmaceutically active agents having contraceptive activity and which are suitable for use in the contraceptive compositions of the present invention include steroid and hormone-derived agents.

As used herein the terms “modulates”/“modulating” preferably mean any one or more of: adversely affecting, decreasing, removing, inhibiting, antagonising, blocking or down regulating EP₂ receptor activity.

The female reproductive system comprises ovaries, fallopian tubes or oviducts, a uterus and vagina. At an early stage in the menstrual cycle and under the influence of gonadotropins, a group of follicles, termed “primary follicles”, is recruited. By day 6 of the menstrual cycle, one of these becomes mature or “dominant”. This process is characterized by enlargement of the fluid-filled antrum and accelerated growth of the granulosa. The follicles which are not destined to ovulate begin degeneration, while the oocyte in the primary follicle is arrested at the prophase of meiosis (Carr and Wilson, 1991).

As used herein the term ‘ovulation’ refers to the release by the follicles of one or more ova or mature oocytes. Ovulation is characterised in humans by specific hormonal changes; in particular, it occurs just subsequent to a surge in luteinising hormone (LH) and follicle stimulating hormone (FSH) levels and a decrease in oestrogen levels.

Just prior to the start of ovulation during the mid-cycle, a preovulatory increase in the gonadotropin LH triggers the final step of maturation (resumption of meiosis) of the oocyte (Tsafriri and Adashi 1996), followed by the rupture of the follicle wall to allow the mature oocyte to be released. A morphological expression of oocyte maturation is the expansion of granulosa cells surrounding the follicle (cumulus expansion) and dissolution of the germinal vesicle (germinal vesicle breakdown, GVBD). If final maturation of the oocyte is prevented or impaired, the oocyte may not be fertilized by a spermatozoon.

Cumulus expansion in the preovulatory follicle may therefore be regarded as an obligate step for successful ovulation and fertilisation, which is regulated by prostaglandin PGE₂, most likely via the EP₂ receptor. The selectivity of this regulatory step has been demonstrated by the observation that expression of the enzyme required for prostaglandin synthesis (cyclooxygenase 2, COX-2) is triggered approximately ten hours prior to ovulation in various mammalian species (Sirois and Dore, 1997).

A further advantage of the contraceptive compositions of the present invention is that the administration of such compositions may be restricted to a relatively short fertile time period. For example, since the compositions act through inhibition of EP₂ receptor-mediated processes on cumulus expansion, ovulation and oogenesis, administration may start at the LH peak and continue during prostaglandin synthesis within the menstrual cycle. Hence, continuous administration of a pharmaceutical contraceptive may be avoided.

As used herein the term, ‘oocyte’ refers to a cell capable of maturing to a female haploid egg cell (ovum) by meiosis. The mature oocyte (ovum) is released at the time of ovulation from the follicles of the ovary.

As used herein the term ‘folliculogenesis’ refers to the process by which oocytes [eggs] enclosed in follicles mature in the ovary and ovulate each cycle. Oocytes in the growing follicle are surrounded by multiple layers of granulosa cells. In late folliculogenesis prior to ovulation, granulosa cells undergo an expansion process mediated by the production and secretion of hyaluronic acid. From the known literature it can be assumed that this cumulus expansion prior to ovulation is a key process for successful ovulation and subsequent fertilisation. Activation of the EP₂ receptor seems to play an important role within this process (Hizaki et al 1999).

In one embodiment of the present invention the antagonist of the present invention is selected from the group consisting of an antagonist, a partial antagonist and a competitive antagonist of an EP₂ receptor.

An antagonist of a given moiety may inhibit one or more activities of that moiety (e.g. all activities). It may, for example, bind in a competitive or non-competitive manner to the moiety or to something with which the moiety interacts (e.g. binds).

The present invention also provides a pharmaceutical composition comprising a therapeutically effective amount of an EP₂ antagonist admixed with a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), adjuvant(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the pharmaceutical composition of the present invention is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the pharmaceutical compositions of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WOA-91/11172, WO-A-94/02518 and WO-A-98/55148.

Pharmaceutical compositions of the present invention may be provided in controlled release form. This can be achieved by providing a pharmaceutically active agent in association with a substance that degrades under physiological conditions in a predetermined manner. Degradation may be enzymatic or may be pH-dependent.

Different drug delivery systems may be used to administer pharmaceutical compositions of the present invention, depending upon the desired route of administration. Drug delivery systems are described, for example, by Langer (Science 249:1527-1533 (1991)) and by Illum and Davis (Current Opinions in Biotechnology 2: 254-259 (1991)).

In a preferred embodiment, the pharmaceutical compositions of the present invention are delivered systemically (such as orally, buccally, sublingually), more preferably orally.

Hence, preferably the pharmaceutical composition is in a form that is suitable for oral delivery.

Preferably, the pharmaceutical compositions of the present invention are provided in a pharmaceutical pack comprising one or more compartments wherein at least one compartment houses an EP₂ receptor antagonist which may be in admixture with one or more of: a COX inhibitor, a pharmaceutically acceptable carrier, diluent or excipient (or combinations thereof), an adjuvant or a pharmaceutically active agent.

The term “administered” includes lipid mediated transfection, liposomes, immunoliposomes, lipofectin, cationic facial amphiphiles (CFAs) and combinations thereof.

Antagonists suitable for use in the present invention may be administered alone but will generally be administered as a pharmaceutical composition—e.g. when the antagonists are in admixture with a suitable pharmaceutical excipient, diluent or carrier selected with regard to the intended route of administration and standard pharmaceutical practice.

For example, the antagonists can be administered (e.g. orally or topically) in the form of tablets, capsules, ovules, elixirs, solutions or suspensions, which may contain adjuvants, flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the antagonist may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal (e.g. by an intrauterine system (IUS) or by drug-containing vaginal rings), intracerebroventricular, intracerebral, subcutaneous, ophthalmic (including intravitreal or intracameral), transdermal, rectal, buccal, vaginal, epidural, sublingual.

It is to be understood that not all of the antagonists need be administered by the same route. Likewise, if the composition comprises more than one active component, then those components may be administered by different routes.

If the antagonists of the present invention are administered parenterally, then examples of such administration include one or more of: intravenously, intra-arterially, intraperitoneally, intrathecally, intraventricularly, intraurethrally, intrasternally, intracranially, intramuscularly or subcutaneously administering the agent; and/or by using infusion techniques.

For parenteral administration, the antagonists are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the antagonists of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the antagonist and a suitable powder base such as lactose or starch.

Alternatively, the antagonists of the present invention can be administered in the form of a suppository or pessary, or it may be applied topically in the form of a gel, hydrogel, lotion, solution, cream, ointment or dusting powder. The antagonist of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isofonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the antagonists of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compositions of the present invention may be administered by direct injection, for some applications, preferably the antagonists are administered orally or topically

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The antagonist and/or the pharmaceutical composition of the present invention may be administered in accordance with a regimen of from 1 to 10 times per day, such as once or twice per day.

Typically, administration of antagonists suitable for use in the contraceptive compositions of the present invention may start at a point in the menstrual cycle prior to ovulation (pre-ovulatory phase).

Preferably, administration of antagonists suitable for use in the contraceptive compositions of the present invention may start at a point in the menstrual cycle when the LH level is at a peak (at which time prostaglandin synthesis is stimulated and late antral follicles are prepared for ovulation).

For oral and parenteral administration to humans, the daily dosage level of the agents may be in single or divided doses.

Depending upon the need, the antagonists may be administered at a dose of from 0.01 to 30 mg/kg body weight, such as from 0.1 to 10 mg/kg, more preferably from 0.1 to 1 mg/kg body weight. Naturally, the dosages mentioned herein are exemplary of the average case. There can, of course, be individual instances where higher or lower dosage ranges are merited.

Typically the daily oral dose may be, for instance, between 20-1000 mg, preferably 50-300 mg for example.

The antagonists suitable for use in the present invention may be formulated into a pharmaceutical composition, such as by mixing with one or more of a suitable carrier, diluent or excipient, by using techniques that are known in the art.

In a preferred aspect, the present invention provides a process for formulating a pharmaceutical composition for subsequent use as a contraceptive comprising admixing an EP₂ receptor antagonist (optionally in combination with a COX inhibitor) and a pharmaceutically acceptable carrier, diluent or excipient (including combinations thereof).

In a further preferred aspect, the present invention provides a process for formulating a pharmaceutical composition for subsequent use as a contraceptive comprising an EP₂ receptor antagonist (optionally in combination with a COX inhibitor) and a pharmaceutically acceptable carrier, diluent or excipient (including combinations thereof); which process further comprises placing said composition in a container.

In a still further preferred aspect of the present invention there is provided a process for formulating a pharmaceutical composition for subsequent use as a contraceptive comprising an EP₂ receptor antagonist (optionally in combination with a COX inhibitor) and a pharmaceutically acceptable carrier, diluent or excipient (including combinations thereof); which process further comprises placing said composition in a container which comprises a label indicating regulatory approval for use as a contraceptive.

As used herein, the term “individual” refers to vertebrates, particularly members of the mammalian species. The term includes but is not limited to domestic animals, sports animals, primates and humans.

Suitably, the contraceptive compositions of the present invention are for administration to a female individual, in particular, a human female.

It is to be appreciated that all references herein to treatment include prophylactic treatment.

Preferably, the contraceptive compositions of the invention (and combinations) are orally bioavailable. Oral bioavailability refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailability.

Dissolution, the solubilisation of the drug by the aqueous contents of the gastrointestinal tract (GIT), can be predicted from in vitro solubility experiments conducted at appropriate pH to mimic the GIT. Preferably the agents of the invention have a minimum solubility of 50 mcg/ml. Solubility can be determined by standard procedures known in the art such as described in Adv. Drug Deliv. Rev. 23, 3-25, 1997.

Membrane permeability refers to the passage of the compound through the cells of the GIT. Lipophilicity is a key property in predicting this and is defined by in vitro Log D_{7.4} measurements using organic solvents and buffer. Preferably the agents of the invention have a Log D_{7.4} of −2 to +4, more preferably −1 to +2. The log D can be determined by standard procedures known in the art such as described in J. Pharm. Pharmacol. 1990, 42:144.

Cell monolayer assays such as CaCO₂ add substantially to prediction of favourable membrane permeability in the presence of efflux transporters such as p-glycoprotein, so-called caco-2 flux. Preferably, antagonists of the invention have a caco-2 flux of greater than 2×10⁻⁶ cms⁻¹, more preferably greater than 5×10⁻⁻⁶ cms⁻¹. The caco flux value can be determined by standard procedures known in the art such as described in J. Pharm. Sci, 1990, 79, 595-600

Metabolic stability addresses the ability of the GIT or the liver to metabolise compounds during the absorption process: the first pass effect. Assay systems such as microsomes, hepatocytes etc are predictive of metabolic liability. Preferably the antagonists of the Examples show metabolic stability in the assay system that is commensurate with an hepatic extraction of less then 0.5. Examples of assay systems and data manipulation are described in Curr. Opin. Drug Disc. Devel., 201, 4, 36-44, Drug Met. Disp.,2000, 28, 1518-1523.

Because of the interplay of the above processes further support that a drug will be orally bioavailable in humans can be gained by in vivo experiments in animals. Absolute bioavailability is determined in these studies by administering the antagonists separately or in mixtures by the oral route. For absolute determinations (% absorbed) the intravenous route is also employed. Examples of the assessment of oral bioavailability in animals can be found in Drug Met. Disp.,2001, 29, 82-87; J. Med Chem, 1997, 40, 827-829, Drug Met. Disp., 1999, 27, 221-226.

In one aspect of the present invention, an EP₂ receptor may be used as a target in screens to identify agents capable of modulating EP₂ receptor activity. By way of example, EP₂ receptor activity may be used as a target in screens to identify agents capable of modulating EP₂ receptor activity such as, for example, impairing cumulus expansion or preventing oocyte maturation.

For convenience, the term “target” as used herein includes a reference to an EP₂ receptor.

Agents for use in the contraceptive compositions according to the present invention may be any suitable agent that can act as an EP₂ receptor antagonist to cause a contraceptive effect. As used herein, the term “agent” includes any entity capable of modulating EP₂ receptor activity.

Preferably the agent is an antagonist.

As used herein, the term “agent” includes, but is not limited to, a compound, such as a test compound, which may be obtainable from or produced by any suitable source, whether natural or not.

The antagonist may be designed or obtained from a library of compounds which may comprise peptides, as well as other compounds, such as small organic molecules and particularly new lead compounds. By way of example, the antagonist may be a natural substance, a biological macromolecule, or an extract made from biological materials such as bacteria, fungi, or animal (particularly mammalian) cells or tissues, an organic or an inorganic molecule, a synthetic test compound, a semi-synthetic test compound, a structural or functional mimetic, a peptide, a peptidomimetics, a derivatised test compound, a peptide cleaved from a whole protein, or a peptides synthesised synthetically (such as, by way of example, either using a peptide synthesizer or by recombinant techniques or combinations thereof, a recombinant test compound, a natural or a non-natural test compound, a fusion protein or equivalent thereof and mutants, derivatives or combinations thereof.

The antagonist of the present invention may also be capable of displaying one or more other beneficial functional properties.

Preferably the antagonist may selectively antagonise, and/or selectively downregulate or selectively inhibit a suitable target.

For some applications, preferably the antagonist has at least about a 5, 10, 25, 50, 75, 100 fold selectivity to the desired target, preferably at least about a 150 fold selectivity to the desired target, preferably at least about a 200 fold selectivity to the desired target, preferably at least about a 250 fold selectivity to the desired target, preferably at least about a 300 fold selectivity to the desired target, preferably at least about a 350 fold selectivity to the desired target.

As used herein, the term “agent” may be a single entity or it may be a combination of agents.

The antagonist can be an amino acid sequence or a chemical derivative thereof. The substance may even be an organic compound or other chemical. The antagonist may even be a nucleotide sequence—which may be a sense sequence or an anti-sense sequence. The antagonist may even be an antibody.

For the purposes of this invention, the term “antibody”, unless specified to the contrary, includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies. Neutralizing antibodies, i.e., those which inhibit biological activity of the substance polypeptides, are especially preferred for diagnostics and therapeutics.

Antibodies may be produced by standard techniques, such as by immunisation with the substance of the invention or by using a phage display library.

An antagonist may act directly or indirectly to provide a desired effect. For example, a cell, vector or nucleic acid may be provided to a patient in order to increase levels of a desired polypeptide (including providing a polypeptide that is not present in the patient). The polypeptide may itself provide the desired effect or may act upon one or more other moieties to provide the desired effect.

In some cases an antagonist may act by inactivating a gene or of preventing or reducing expression at the RNA or polypeptide level. For example “knock-out” techniques may be used to render certain genes non-functional. Antisense techniques may be used to block RNA production or translation.

An antagonist of the present invention may be provided in substantially pure or substantially isolated form. It may be provided in the form of a pharmaceutically acceptable composition—e.g admixed with a suitable pharmaceutically acceptable carrier, diluent, or excipient.

The antagonist of the present invention may be in the form of, and/or may be administered as, a pharmaceutically acceptable salt such as an acid addition salt or a base salt, or a solvate thereof, including a hydrate thereof. For a review on suitable salts see Berge et al, J. Pharm. Sci., 1977, 66, 1-19.

Typically, a pharmaceutically acceptable salt may be readily prepared by using a desired acid or base, as appropriate. The salt may precipitate from solution and be collected by filtration or may be recovered by evaporation of the solvent.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc and diethanolamine salts.

The present invention encompasses a range of screening methods.

Any one or more of appropriate targets—such as an amino acid sequence and/or nucleotide sequence—may be used for identifying an agent, e.g. an EP₂ receptor antagonist, in any of a variety of drug screening techniques. The target employed in such a test may be free in solution, affixed to a solid support, borne on a cell surface, or located intracellularly. The target may even be within an animal model, wherein said target may be an exogenous target or an introduced target. The animal model will be a non-human animal model. The abolition of target activity or the formation of binding complexes between the target and the agent being tested may be measured.

Techniques for drug screening may be based on the method described in Geysen, European Patent Application 84/03564, published on Sep. 13, 1984. In summary, large numbers of different small peptide test compounds are synthesised on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

Alternatively, animal models or computer models may be used. Non-human animals (e.g. primates) already possessing the targets described herein may be used. Alternatively, transgenic nonhuman animals (e.g. rodents) capable of expressing the EP₂ receptor may be produced. Techniques for producing transgenic animals are well known and are described e.g. in US-A-4870009 and US-A4873191. For example, a nucleic acid encoding a desired polypeptide may be microinjected into a pronucleus of a fertilised oocyte. The oocyte may then be allowed to develop in a pseudopregnant female foster animal. The animal resulting from development of the oocyte can be tested (e.g. with antibodies) to determine whether or not it expresses the polypeptide. Alternatively, it can be tested with a probe to determine if it has a transgene. A transgenic animal can be used as a founder animal, which may be bred from in order to produce further transgenic animals. Two transgenic animals may be crossed. For example, in some cases transgenic animals may be haploid for a given gene and it may be desired to try to provide a diploid offspring via crossing. A transgenic animal may be cloned, e.g. by using the procedures set out in WO-A-97/07668 and WO-A97/07699 (see also Nature 385:810-813 (1997)). Thus a quiescent cell can be provided and combined with an oocyte from which the nucleus has been removed combined. This can be achieved using electrical discharges. The resultant cell can be allowed to develop in culture and can then be transferred to a pseudopregnant female.

Test agents capable of modulating EP₂ receptor activity may be screened in assays well-known in the art. Screening can be, for example in vitro, in cell culture, and/or in vivo. Biological screening assays preferably centre on activity-based response models, binding assays (which measure how well an agent modulates receptor activity), and bacterial, yeast and animal cell lines (which measure the biological effect of a compound in a cell). The assays can be automated for high capacity-high throughput screening (HTS) in which large numbers of compounds can be tested to identify compounds with the desired activity (see for example WO 84/03564). The biological assay, may also be an assay for antagonist binding activity of a compound that selectively binds to the EP₂ receptor compared to other prostanoid receptors. Once an agent capable of modulating the receptor activity has been identified, further steps may be carried out either to select and/or to modify compounds and/or to modify existing compounds, to improve the receptor activity modulation capability.

Specific screens may be based upon the mode of action of EP₂ receptors.

Screening may be performed in vitro. This may be done for example by using oocytes or ovaries or parts of both thereof. For example, cumulus expansion may be investigated in vitro using the assay method of Vanderhyden et al, 1990.

In one aspect, the present invention provides an in vitro assay for identifying putative EP₂ receptor antagonists comprising the use of cumulus enclosed oocyte (CEO) complexes. The CEO complexes may be obtained from mouse ovaries after stimulation of folliculogenesis.

It is proposed that this model may be useful for the selection of EP₂ receptor antagonists capable of suppression of EP₂ receptor-mediated cumulus expansion.

In a further aspect, the present invention provides an ex vivo assay for identifying putative EP₂ antagonists comprising the use of ovarian perfusion assays.

In addition, putative EP₂ receptor antagonists may also be tested in in vivo test models to investigate their effects on ovulation, cumulus expansion, fertilisation, implantation and pregnancy rate to assess their potential as contraceptive agents.

In a yet further aspect of the present invention there is provided an in vivo assay for identifying putative EP₂ antagonists comprising administration of a putative EP₂ antagonist to a sexually mature or immature subject and measuring the resultant effects therein on ovulation, cumulus expansion, fertilisation, implantation and pregnancy rate.

Another technique for screening provides for high throughput screening (HTS) of agents having suitable binding affinity to EP₂ receptors and is based upon the method described in detail in WO 84/03564.

By way of further example, screens may be based upon binding studies. Here, binding agents to EP₂ receptors or to moieties operatively associated with them (e.g. moieties capable of mediating EP₂ receptor activity) may be useful as receptor antagonists. Binding studies are therefore useful in identifying agents that may then be subjected to further screening as discussed above. Such studies include providing a binding agent and determining whether or not it binds to an EP₂ receptor or to a moiety operatively associated with the receptor. If desired, the binding agent may be labelled to aid in identification.

In addition to various screening methods of the present invention, an agent identified or identifiable by such a screening method (of whatever nature) is within the scope of the present invention. This may itself be useful as a therapeutic agent or may be used in a drug development program leading to the provision of a therapeutic agent.

It is expected that the assay methods of the present invention will be suitable for both small and large-scale screening of test compounds as well as in quantitative assays.

Thus, the present invention provides a method of identifying agents capable of modulating EP₂ receptor activity in an individual, the method comprising contacting a suitable target from (or obtainable from) an individual and then measuring the activity of the receptor.

The present invention also relates to a method of identifying agents that modulate EP₂ receptor activity the method comprising contacting a suitable target with the agent and then measuring the activity and/or levels of expression of the receptor.

The present invention also relates to a method of identifying agents that selectively modulate EP₂ receptor activity the method comprising contacting a suitable target with the agent and then measuring the activity and/or levels of expression of the receptor.

In a preferred aspect the present invention relates to an assay comprising identifying agents that may be used in a contraceptive composition, the assay comprising; determining whether an agent can directly or indirectly modulate EP₂ receptor activity; wherein modulation of EP₂ receptor activity in the presence of the agent is indicative that the agent may be useful as a contraceptive; and wherein said agent is an EP₂ receptor antagonist (optionally in combination with one or more COX inhibitors).

The diagnostic compositions and/or methods and/or kits may be used in the following techniques which include but are not limited to; competitive and non-competitive assays, radioimmunoassay, bioluminescence and chemiluminescence assays, fluorometric assays, sandwich assays, immunoradiometric assays, dot blots, enzyme linked assays including ELISA, microtiter plates, antibody coated strips or dipsticks for rapid monitoring of urine or blood, immunohistochemistry and immunocytochemistry.

In vivo models may be used to investigate and/or design therapies or therapeutic agents for use as contraceptives. The models could be used to investigate the effect of various tools/lead compounds on a variety of parameters which indicate EP₂ receptor modulation, for example impaired cumulus expansion.

The invention further provides transgenic nonhuman animals capable of expressing the nucleotide sequence encoding an EP₂ receptor or a variant, homologue, derivative or fragment thereof for use in the present invention and/or a transgenic nonhuman animal having one or more nucleotide sequence encoding an EP₂ receptor for use in the present invention or a variant, homologue, derivative or fragment thereof inactivated. Expression of such a nucleotide sequence is usually achieved by operably linking the nucleotide sequence to a promoter and optionally an enhancer, and microinjecting the construct into a zygote. See Hogan et al., “Manipulating the Mouse Embryo, A Laboratory Manual,” Cold Spring Harbor Laboratory. Inactivation of such a nucleotide sequence may be achieved by forming a transgene in which a cloned nucleotide sequence is inactivated by insertion of a positive selection marker. See Capecchi, Science 244, 1288-1292 (1989). The transgene is then introduced into an embryonic stem cell, where it undergoes homologous recombination with an endogenous variant gene. Mice and other rodents are preferred animals. Such animals provide screens and/or screening systems for identifying agents capable of modulating receptor activity.

The present invention will now be described by way of example only in which reference may be made to the following:

FIG. 1, which shows a schematic pathway of the biosynthesis of prostaglandins.

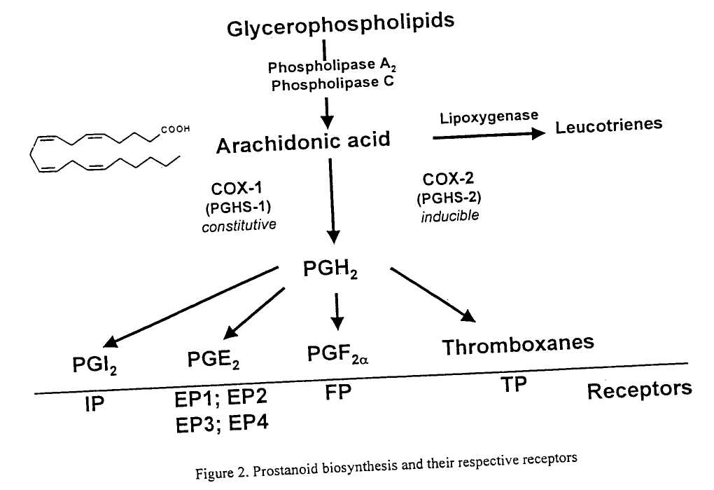

FIG. 2, which shows prostanoid biosynthesis and their respective receptors.

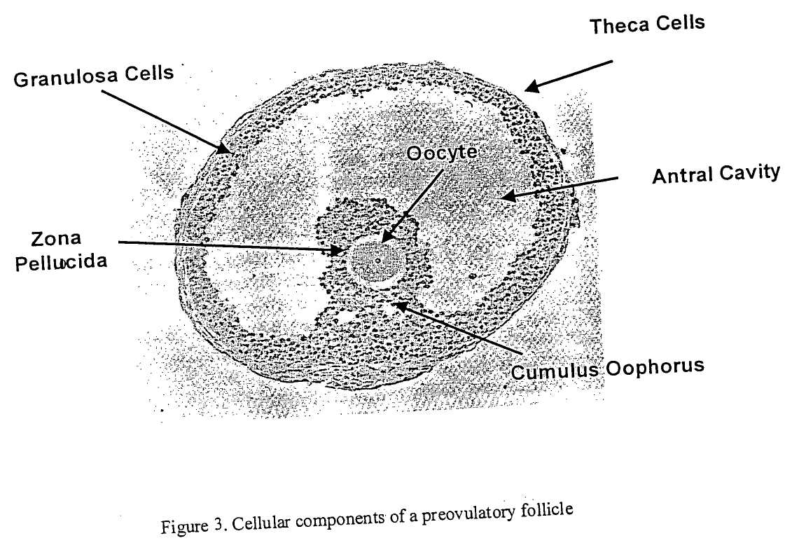

FIG. 3, which shows the cellular components of a preovulatory follicle.

FIG. 4, which shows the influence of AH 6809, an EP2 antagonist, on the cumulus expansion in vitro. Cumulus oocyte complexes (COC) were cultured in MEMa medium with Fetal Bovine Serum (10%), IBMX (100 μM) and glucosamine (2.5 mM); n=11-12 COC per group.

All groups contained the same amount of ethanol. 1) vehicle control; 2) FSH (0.002 U); 3) FSH (0.02 U); 4) PGE2 (1 μM); 5) PGE2 (1 μM)+AH 6809 (10 μM); 6) PGE2 (1 μM)+AH 6809 (50 μM); 7) PGE2 (1 μM)+AH 6809 (100 μM); 8) PGE2 (1 μM)+AH 6809 (200 μM)

### EXAMPLES

1. Preparation of Cumulus Enclosed Oocytes

Cumulus Enclosed Oocytes (CEO's) are obtained from immature female mice (C57BL/6J×DBA/2JF1) weighing 13-16 grams, kept under controlled temperature (20-22° C.), light (lights on 06.00-18.00) and relative humidity (50-70%). The mice receive an intra-peritoneal injection of 0.2 ml gonadotropins containing 20 IU FSH and 48 hours later the animals are killed by cervical dislocation. The ovaries are dissected out and the CEO's are isolated under a stereo microscope by manual rupture of the follicles using a pair of 27 gauge needles. Cumulus enclosed oocytes displaying an intact germinal vesicle are placed in a minimum essential medium (MEM) without ribonucleosides supplemented with 8 mg/ml Human Serum Albumin (HAS), 0.23 mM pyruvate, 2 mM glutamine, 100 lU/ml penicillin and 100 μg/ml streptomycin. These complexes are cultured for 20 h in the presence of FSH (Follicle Stimulating Hormone) with or without test compounds. Thereafter cumulus expansion is assessed according to 5 stages (0=no response, +4=maximal response) (FIG. 4, Vanderhyden et al. 1990 and Hizaki et al. 1999).

2. Ex Vivo Perfusion of Rat Ovary

Immature rats are injected with PMSG 48 hours prior to ovary preparation for organ perfusion (Hegele-Hartung et aL 2001). Ovulation is induced in the perfused ovary by oLH (ovine Lutenizing Hormone) with or without simultaneous perfusion of test compounds. After perfusion for 20 hours, the number of ovulated oocytes with or without germinal vesicle breakdown (GVB) is assessed.

3. Effects on Ovulation in Immature Rodents

Immature mice are treated with PMSG to stimulate follicle growth (day 1). On day 3 animals are treated with hCG (human Chorion Gonadotropin) to induce ovulation and the test compounds are administrated simultaneously p.o or s.c. Mice are killed approximately 1 day after hCG treatment and the number of ovulated oocytes in the oviduct are determined. Blood samples are taken for hormone analysis. (The ovary is fixed in formalin for Histological analysis).

The following parameters are determined:

% animals with inhibition of ovulation compared to vehicle controls.

hormone levels of sex steroids and gonadotropins

histological analysis of the ovary (optional)

Similar experiments may be performed with rats.

4. Effects on Ovulation in Adult Rodents

Mice within the metestrus stage (day 1) are stimulated with male urine/cage spread from male animals to proceed to the oestrus stage. Beginning with this stimulation test compounds are dosed p.o. or s.c. for 5 days. Vaginal smears are performed every day and animals in the oestrus stage are killed 24 hours later in the metestrus stage and the number of ovulated oocytes in the oviduct are determined. Blood collections are performed to evaluate hormone.

Similar experiments with rats may be performed.

5. Fertility Tests in Adult Rodents

Adult mice are treated for one cycle with test compounds. After mating, the number of gravid animals, live embryos, dead embryos and implantation sites are determined on day 8 after conception and compared to a placebo treated group.

Similar experiments with rats may be performed.

Inhibition of the effects on late folliculogenesis, which are mediated by the EP₂ receptors, by administration of an EP₂ receptor antagonist (optionally in combination with one or more COX inhibitors), impairs cumulus expansion and oocyte maturation. Thus, it is now known that administration of an EP₂ receptor antagonist (optionally in combination with one or more COX inhibitors) prevents ovulation or provides an oocyte which, when released, may not have the competence to be fertilised. Thus, by deactivating an EP₂ receptor by administration of an EP₂ receptor antagonist (optionally in combination with one more COX inhibitors), ovulation can be impaired such that fertilisation of an oocyte is not possible and thus pregnancy may be prevented.

Thus, an EP₂ receptor antagonist (optionally in combination with one or more COX inhibitors) can be used in contraceptive compositions.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or biology or related fields are intended to be covered by the present invention. All publications mentioned in the above specification are herein incorporated by reference.

### REFERENCES

1) Breyer R. M., Bagdassarian C. K., Myers S. A. et al. (2001) Prostanoid Receptors: Subtypes and Signaling. Annu. Rev. PharmacoL Toxicol. 41, 661-690.

2) Davis B. J., Lennard D. E., Lee, C. A. et al. (1999) Anovulation in cyclooxygenase-2-deficient mice is restored by prostaglandin E₂ and interleukin-1β. Endocrinology, 140, 2685-2695.

3) Duffy D. M. and Stouffer R. L. (2001) The ovulatory gonadotrophin surge stimulates cyclooxygenase expression and prostaglandin production by the monkey follicle. Mol Hum Reprod. 7, 731-739.

4) Hegele-Hartung C., Grutzner M., Lessl M. et al. (2001) Activation off meiotic maturation in rat oocytes after treatment with follicular fluid Meiosis-Activating Sterol in vitro and ex vivo. Biol. Reprod. 64, 418-424.

5) Hizaki H, Segi E, Sugimoto Y., et al.(1999) Abortive expansion of the cumulus and impaired fertility in mice lacking the prostaglandin E receptor subtype EP₂. Proc Natl Acad Sci USA; 96, 10501-10506

6) Kennedy C. R. J., Zhang Y., Brandon S. et al. (1999) Salt-sensitive hypertension and reduced fertility in mice lacking the prostaglandin EP₂ receptor. Nat Med 5, 217-220.

7) Killick S. and Elstein M. (1987) Pharmacologic production of luteinized unruptured follicles by prostaglandin synthetase inhibitors. Fertil. Steril., 47, 773-777.

8) Langenbach R., Morham S. G., Tiano, H. F. et al. (1995) Prostaglandin synthase 1 gene disruption in mice reduces arachidonic acid-induced inflammation and indomethacin-induced gastric ulceration. Cell, 83, 483-492.

9) Lim H., Paria B. C., Das S. K. et al. (1997) Multiple female reproductive failures in cyclooxygenase 2-deficient mice. Cell, 91, 197-208.

10) Marks F. & Furstenberger G. (Eds.); Prostaglandins, Leukotrienes and other Eicosanoids. Wiley-VCH 1999, Chapter 8

11) Matsumoto H, Ma W, Smalley W, Trzaskos J, et al. (2001) Diversification of cyclooxygenase-2-derived prostaglandins in ovulation and implantation. Biol Reprod. 64, 1557-1565.

12) Mikuni M., Pall, M., Peterson, C. M. et al. (1998) The selective prostaglandin endoperoxide synthase-2 inhibitor, NS-398, reduces prostaglandin production and ovulation in vivo and in vitro in the rat. Biol. Reprod., 59, 1077-1083.

13) Murdoch W. J. (1996) Differential effects of indomethacin on the sheep ovary: prostaglandin biosynthesis, intracellular calcium, apoptosis, and ovulation. Prostaglandins, 52, 497-506.

14) Norman R. J. (2001) Reproductive consequences of COX-2 inhibition. Lancet, 358, 1287-1288

15) Pall M., Friden B. E., and Brannstrom M. (2001) Induction of delayed follicular rupture in the human by the selective COX-2 inhibitor rofecoxib: a randomized double-blind study Hum Reprod, 16, 1323-1328.

16) Richards J. S. (1994) Hormonal control of gene expression in the ovary. Endocr. Rev., 15, 725-751.

17) Salhab A. S., Gharaibeh M. N., Shomaf M. S, et al. (2001) Meloxicam inhibits rabbit ovulation. Contraception, 63, 329-333.

18) Sirois, J. and Dore, M. (1997) The late induction of prostaglandin G/H synthase-2 in equine preovulatory follicles supports its role as a determinant of the ovulatory process. Endocrinology, 138, 4427-4434.

19) Tilly SL, Audoly LP, Hicks EH, et al. (1999) Reproductive failure and reduced blood pressure in mice lacking the EP₂ prostaglandin E₂ receptor. J Clin Invest; 103, 1539-1545.

20) Vanderhyden BC, Caron BJ, Buccione R et al. (1990) Developmental pattern of the secretion of cumulus expansion-enabling factor by mouse oocytes and the role of oocytes in promoting granulosa cell differentiation. Dev. Biol., 140, 307-317

21) Wallach E. E., de la Cruz A., Hunt, J. et al. (1975) The effect of indomethacin on hMG-hCG induced ovulation in the rhesus monkey. Prostaglandins, 9, 645-658.

22) Woodward DF, (1995) 6-Isopropoxy-9-oxoxanthene-2-carboxylic acid (AH 6809), a human EP2 receptor antagonist. Biochem. Pharm. 50, 1731-1733

23) Zanagnolo V., Dharmarajan A. M., Endo K. et al. (1996) Effects of acetylsalicylic acid (aspirin) and naproxen sodium (naproxen) on ovulation, prostaglandin, and progesterone production in the rabbit. Fertil. Steril., 65, 1036-1043.

1. A method for impairing cumulus expansion and oocyte maturation, comprising antagonizing the EP₂ receptor and/or inhibiting cyclooxygenase COX-2.

2. The method of claim 1, wherein the EP₂ receptor is antagonised by the EP₂ receptor antagonist AH6809.

3. The method of claim 1, wherein said EP₂ receptor antagonist is used in a composition and wherein said composition further comprises one or more COX inhibitors.

4. A method of fertility control in a woman comprising administering an EP₂ receptor antagonist and/or a cyclooxygenase COX-2 inhibitor.

5. The method according to claim 4, wherein said EP₂ receptor antagonist is used.

6. The method according to claim 5, wherein said EP₂ receptor antagonist is AH6809 and is used in a composition and wherein said composition further comprises one or more COX inhibitors.

7. The method according to claim 6, wherein said COX inhibitor is selective for cyclooxygenase-2 (COX-2).

8. The method according to claim 6 wherein the COX inhibitor is selected from the group consisting of Celecoxib, Ceracoxib, Parecoxib, Rofecoxib, Valdecoxib, NS-398 and Etoricoxib.

9. The method according to claim 6 wherein the COX inhibitor is Celecoxib or Rofecoxib.

10. The method according to claim 6 wherein said EP₂ receptor antagonist is used and is in a form suitable for oral delivery.

11. The method of claim 1 for achieving fertility control in a woman.
